(19) United States
(12) Patent Application Publication
(10) Pub. No.: US 2007/0099177 A1
(43) Pub. Date: May 3, 2007

(54) Agents and methods for detecting human adenoviruses

(76) Inventors: Albert Roland Heim, Hannover (DE); Patricia Pring-Akerblom, Hannover (GB)

Correspondence Address: AKERMAN SENTERFITT, P.O. BOX 3188, WEST PALM BEACH, FL, 33402-3188, US

(21) Appl. No.: 10/534,990

(22) PCT Filed: Nov. 14, 2002
(86) PCT No.: PCT/EP02/12756

§ 371 Date: Nov. 9, 2006

(51) Int. Cl. C12Q 1/70; C12Q 1/68; C12P 19/34; C07H 21/04

(52) U.S. Cl. 435/5; 435/6; 435/91.2; 536/24.1

(57) ABSTRACT

The invention relates to labeled or unlabelled nucleic acids to specifically bind to DNA of human adenoviruses (HAdV DNA), whereby the nucleic acid a) possesses the sequence SEQ ID NO. 1, SEQ ID NO. 2, or SEQ ID NO. 3, b) possesses a sequence with a homology of greater than 78% with respect to SEQ ID NO. 1, SEQ ID NO. 2, or SEQ ID NO. 3, or c) is complementary with respect to a nucleic acid according to a) or b). Also described are methods for the detection of HAdV DNA.

### CROSS-REFERENCES TO RELATED APPLICATIONS

This application is a national stage of PCT/EP/2002/012756 filed Nov. 14, 2002.

### BACKGROUND OF THE INVENTION

1. Field of the Invention

The present invention relates to primers, probes, as well as a kit thereof for the detection of human adenoviruses. The invention further relates to detection techniques that are able to identify the DNA of 15 or more HAdV serotypes.

2. Description of the Related Art

The 6 species (old: subgenera) of human adenoviruses (HAdV) with their 51 serotypes are associated with a multitude of diseases that can affect all organs (see Wadell, G., A. Allard, and H. Hierholzer. 1999. Adenovirus, p. 970-981. In P. R. Murray, E. J. Baron, M. A. Pfaller, C. A. Tenover, and R. A. Yolken (ed.), Manual of Clinical Microbiology. ASM Press, Washington, D.C.). Examples of such diseases are acute diseases of the respiratory tract in infants and small children, severe pneumonia, pharyngoconjunctival fever (PCF), epidemic keratoconjunctivitis (EKC), suppurating genital lesions, cervicitis, gastroenteritis and urethritis. Especially in immunosuppressed patients, such as for example recipients of organ transplants and bone marrow transplants, initially latent infections of the adenoids or the urogenital tract can lead to a significant load of HAdV of various serotypes.

The usual method to diagnose HAdV infections involves isolating the virus with subsequent typing. But it may take up to three weeks for any cytopathic effects to develop and some HAdV types are cultivated slowly and inefficiently or require special cell lines such as 293 Graham cells for the isolation process. For these reasons, many research groups have developed PCR protocols to detect HAdV in clinical samples. Most of these PCRs were created as “generic protocols” to detect as many types of the genus HAdV as possible (Allard, A., B. Albinsson, and G. Wadell. 2001. Rapid typing of human adenoviruses by a general PCR combined with restriction endonuclease analysis. J Clin Microbiol. 39(2): 498-505; Echavarria, M., M. Forman, J. Ticehurst, J. S. Dumler, and P. Charache. 1998. PCR method for detection of adenovirus in urine of healthy and human immunodeficiency virus-infected individuals. J Clin Microbiol. 36(11):3323-6; Pring-Akerblom, P., and T. Adrian. 1994. Type- and group-specific polymerase chain reaction for adenovirus detection. Res Virol. 145(1):25-35).

However, the listed publications either disclose only the means and methods to detect a small number of HAdV serotypes or describe the use of degenerate primers in the PCR, which in fact means the use of a large number of primer pairs, each of which will only specifically bind to a small number of HAdV serotypes in the described systems. Not disclosed is a probe for the detection of HAdV DNA.

In the present text we will adhere to the following definitions:

Primer: Short DNA or RNA oligonucleotide, which represents the starting point of the DNA synthesis during the polymerase chain reaction (PCR) and whose base sequence can be completely described for every position of the sequence by specifying one of the respective bases adenine, cytosine, guanine, and thymin or uracil.

Degenerate Primer: Mixture of primers, which in any textual representation are usually condensed into a single sequence by inserting variables at the positions of the sequence where the individual primers of the mixture differ with respect to each other, whereby the variables can represent any of the bases present in the primer mixture for the respective position of the sequence.

Primer pair: Two primers; of which each one can specifically bind to one of the two DNA strands of a DNA, so that the section of the DNA located between these two primers (including the sections bound to the primers) can be amplified by means of a PCR.

Probe: Nucleic acid sequence, which in a single-stranded and usually labeled form is enabled to hybridize specifically to a complementary sequence or sequences similar to the complementary sequence and therefore allows the qualitative or quantitative detection of these sequences.

Specific binding: Hybridization of a probe or a primer to a nucleic acid with no more than 20% mismatches between the bases of the probe or the primer and the nucleic acid.

Mismatches: Base pairs that are not formed by a combination of (a) cytosine (C) and guanine (G) or (b) adenine (A) and thymin (T) or (c) adenine (A) and uracil (U) (the latter in the case of DNA/RNA hybrids).

All or more precisely all known HAdV Serotypes: The HAdV serotypes according to N.N. (2000): Adenoviridae, pp. 227-238. In: M. H. V. Van Regenmortel, C. M. Fauquet, D. H. L. Bishop, E. B. Carsten, M. K. Estes, S. M. Lemon, J. Maniloff, M. A. Mayo, D. J. McGeoch, C. R. Pringle, and R. B. Wickner (Eds): Virus Taxonomy. Seventh Report of the International Committee for the Taxonomy of Viruses, Academic Press, New York, San Diego.

Homology: Term for the degree of similarity between two DNA sequences or between one DNA sequence and one RNA sequence. The degree of homology (in %) corresponds to the degree (in %) of identity as determined by a comparison of the two sequences using the programme EMBOSS::needle (global) (Settings: Gap Open: 10.0; Gap Extend: 0.5; Molecule: DNA; Matrix: DNAfull). (This program implements the Needleman and Wunsch aligning algorithm; see Needleman, S. B. and Wunsch, C. D. (1970), A general method applicable to the search for similarities in the amino acid sequence of two proteins. J. Mol. Biol. 48, 443-453.) To compare RNA sequences with DNA sequences, one replaces any U (uracil) by T when entering the RNA sequence.

Complementary: The sequences of two nucleic acids are complementary if they can be hybridized together without any mismatches, whereby A-T and A-U pairs in DNA/RNA hybrids are not seen as a mismatch.

Quantitative detection: Determining the concentration of the DNA of HAdV viruses under investigation in samples; at least a relative concentration with respect to each other. Ideally the quantitative detection (quantification) also allows conclusions to be drawn about the absolute concentration of said DNA.

Characterization: Carrying out one or several information-gathering steps, which (a) allows assigning the DNA to its organism or virus of origin or (b) yields the result that the state of technology does not yet contain sufficient information to assign the DNA to its organism or virus of origin.

The high sequence diversity of HAdV represents a technical problem that presents enormous difficulty to experts in the fields of medicine and molecular biology when attempting to simultaneously detect DNA material that may originate from a large number of HAdV serotypes. This is so because this diversity makes it almost impossible to find regions in the genomes of the HAdV serotypes that allow the specific binding of individual PCR primers and/or probes to a large number of HAdV DNA sequences of different serotypes.

### SUMMARY OF THE INVENTION

The primary problem to be solved by the present invention was (a) to find primers and/or (b) probes, which (a) allow specific amplification and/or (b) the specific identification of the DNA of a large number of different HAdV serotypes.

This problem is solved by the invention through the use of labeled or unlabelled nucleic acids that bind specifically to DNA of human adenoviruses (HAdV DNA), whereby each nucleic acid

a) possesses the sequence SEQ ID NO. 1, SEQ ID NO. 2, or SEQ ID NO. 3,

b) possesses a sequence with a homology of greater than 78% with respect to SEQ ID NO. 1, SEQ ID NO. 2, or SEQ ID NO. 3, or

c) is complementary with a nucleic acid according to a) or b).

### BRIEF DESCRIPTION OF THE DRAWINGS

FIG. 1 represents the multiple alignment of a portion of the hexon gene sequences of various HAdV serotypes, the consensus sequences of the PCR primers (AQ1, SEQ ID NO. 1; AQ2, SEQ ID NO. 2), and of the probe AP (SEQ ID NO. 3). The melting temperatures (Tₘ of the bond of AQ1 and AQ2 as well as AP) to each sequence were determined taking into account the mismatches between the consensus sequence and the respective HAdV sequence. The base numbering is based on the HAdV-2 sequence. The database numbers are: HAdV-2 (#NC 001405), HAdV-3 (#X76549), HAdV-4 (#AF06062), HAdV-5 (#NC 00146), HAdV-12 (#AF065065), HAdV-34 (#AB052911), HAdV-40 (#L19443), HAdV-41 (#M21163).

FIG. 2 shows the rise in fluorescence as a function of cycle number. Tested were three different template concentrations (A, B: 1.5×10⁷ copies per run, C, D: 1.5×10⁴ copies per run; E, F: 1.5×10¹ copies per run) with (B, D, F) and without (A, C, E) 500 ng of human DNA. The rise of the fluorescence above the threshold value (Crossing Point, CP) was not affected by the human DNA, whereas the final fluorescence value was.

### DETAILED DESCRIPTION OF THE INVENTION

A person skilled in the art has several options for labelling the nucleic acid, such as for example fluorescent, luminescent, ink, or radioactive labels, or enzymes that catalyze the formation of detectable reaction products, or solids, for example metal particles such as magnetic bits.

A surprising result produced by the use of primers of SEQ ID NO. 1 and SEQ ID NO. 2 as primer pair in a PCR was the amplification of DNA of all HAdV serotypes owing to the specific binding of the primers. It was even more surprising to find that in the region framed by the primers, the DNA of each serotype contained a section to which a probe with SEQ ID NO. 3 (or a sequence complementary to SEQ ID No. 3) can specifically bind. Furthermore, it has been found that if the mentioned nucleic acids are used as primer and probe under stringent conditions (≧50° C., preferably ≧55° C.), they are able to specifically bind to the DNA of 45, preferably to the DNA of all of the presently known HAdV serotypes.

Just as surprising is the fact that—despite the high sequence diversity of the DNA of HAdV serotypes—the three conserved regions—to which the above-mentioned primers and probes bind—are located in a comparatively short range of less than 150 bases for all of the HAdV serotypes sequenced to date.

Accordingly, a combination of the invention's nucleic acids in the form of primers of SEQ ID NO. 1 and SEQ ID NO. 2 and a probe of SEQ ID NO. 3 (or a complementary sequence) is a particularly practical ensemble for the detection of the DNA of HAdV.

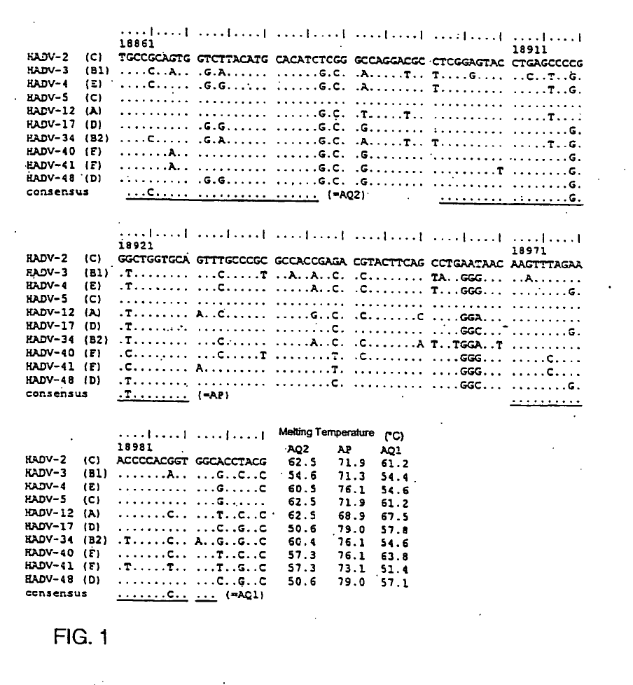

Also surprising was the finding that in practical application the mentioned primers and the probe were able to bind even at annealing temperatures higher than the calculated theoretical melting temperature of the primers with their binding sections attached to the DNA of individual HAdV serotypes, and consequently allowed a specific amplification of the DNA of these serotypes and detection of this DNA (see FIG. 1, multiple alignment, as well as examples).

Of course, in some cases it may be practical to create primer pairs out of a combination of the invention's nucleic acid with SEQ ID NO. 1 and the one with SEQ ID NO. 3 or a combination of SEQ ID NO.2 and the sequence complementary to SEQ ID NO. 3, in particular if the sole objective is the amplification of the DNA of all HAdV serotypes.

It is also possible (using appropriate labelling) to use the invention's nucleic acids with SEQ ID NO. 1 and SEQ ID NO. 2 as well as the complementary nucleic acids as specific probes for the detection of DNA of all HAdV serotypes.

In practical medical applications it often is not necessary to amplify or detect the DNA of all HAdV serotypes. Experiments, theoretical considerations, and computations in this area have shown that according to the invention nucleic acids with a sequence showing a homology >78% with respect to SEQ ID NO. 1, SEQ ID NO. 2, or SEQ ID NO. 3 are still able to specifically bind to a large number of the DNA of the HAdV serotypes. For example, in the case of the SEQ ID NO. 1, a homology of >78% means that the homologous sequence can differ from the SEQ ID NO. 1 in up to 5 positions of the base sequence. Of course, nucleic acids according to the invention that have a greater homology with respect to the sequences of the mentioned SEQ IDs, for example >82%, >86%, >91%, and >95% are preferred, whereby each of the listed percentages allows one less mismatch with respect to SEQ ID NO. 1.

The invention's nucleic acids are preferably selected as probe or primer in a way to be able to specifically bind to the DNA of ≧15, ≧25, ≧30, ≧35, ≧40, or even ≧45 HAdV serotypes. A person skilled in the art can determine this capability in experiments or in a database-based sequence comparison between a nucleic acid sequence according to the invention and the DNA of individual HAdV serotypes, whereby the sequence comparison may include all those HAdV serotypes with known sequences in the area of the binding region of the nucleic acid according to the invention (the hexon gene).

Also part of the invention is a method to determine the nucleic acids that as primers and probes (as well) can specifically bind to a large number of the DNA of various HAdV serotypes:

For this purpose, in a first step one analyzes the genetic variability of various regions of the genomes of all the HAdV completely sequenced to date by multiple alignment of these genome data.

In a second step, one finds one or several highly conserved sections with a length of approximately 20 base pairs. In a preferred embodiment, one determines three highly conserved sections in a region with a length of less than 1000 (preferably less than 500, even more preferred less than 200) base pairs.

A third step consists of a re-analysis of the determined genome section or sections or the determined region containing the three highly conserved sections using a second multiple alignment, which preferably takes into account all available HAdV sequencing data, i.e. even the known data of HAdV serotypes that have not been completely sequenced to date.

In a final step, one determines, with the help of the second multiple alignment, one, two, or preferably three consensus sequences, which are chosen—by calculating the melting temperatures for the hybridization of the primer sequences and probe sequences with the known sequences of the multiple alignment—so that one can be confident of an effective specific binding of the probe and primer to the DNA of the above-mentioned large number of HAdV serotypes.

The invention further concerns a method to detect HAdV DNA in a sample, comprising the following steps:

Providing a sample that may contain HAdV DNA,

Providing a probe, which can specifically bind to the DNA of at least 35 different HAdV serotypes,

Mixing the probe with the sample,

Amplifying regions of the DNA of the up to 35 HAdV serotypes actually present in the sample, so that the section to which the mentioned probe can specifically bind will be amplified as well,

Establishing conditions that allow the probe to specifically bind to sections of the amplified regions,

Detecting amplified DNA sections to which a probe has bound.

The probe is preferably chosen to specifically bind to the DNA of more than 35 HAdV serotypes, i.e. to the DNA of ≧40, more preferable of ≧45, and even more preferable of all HAdV serotypes.

Preferably one uses as probe for this purpose a nucleic acid according to the invention, in particular one with the sequence (a) of SEQ ID NO. 3 (b), with a sequence with a homology >78% with respect to SEQ ID NO. 3, or (c) with a sequence complementary to (a) or (b).

The sample that is to be provided and may possibly contain HAdV DNA is prepared using methods known to a person skilled in the art from clinical or other samples, such as cell cultures, blood, plasma, serum, stool, sputum, urine, eye smears or pharyngonasal smears, or cerebrospinal fluid (CSF), and is to be used in a DNA amplification process, preferably a PCR. A person skilled in the art can easily establish the conditions necessary for specific binding of the respective probes by varying appropriate parameters, in particular by varying the temperature.

According to the invention, the amplification of DNA in most cases will be carried out using the PCR method. But it can also be performed in other ways, e.g. by virus propagation or vector cloning.

Depending on the choice of the labelling of the probe and the probe itself, the detection of the amplified DNA sections that a probe has bound to can be performed using methods such as northern blot, western blot, chemiluminescence, or fluorescence.

It must be emphasized that the order of the steps of the method according to the invention can be adapted to the given requirements and that individual or sequences of steps may be repeated, even several times if necessary. The latter applies especially to steps typical of a PCR. In many cases—depending on the detection method—the mixing of the probe with the sample will only take place after the amplification step/s.

The invention's method offers the advantage, that a single probe makes it possible to prove that DNA from a group of 35 (or ≧40 or ≧45 or all) HAdV serotypes is present in the sample under investigation and consequently in the clinical sample from which the sample under investigation was obtained. In particular, the probe can be used to characterize the amplified DNA regions in more detail and in this way to differentiate these DNA regions from other DNA regions (unintentionally) amplified as well (e.g. from pseudogenes or due to poor primer selection).

A further method according to the invention relates to the detection of HAdV DNA in a sample and comprises the following steps:

Providing a sample that may contain HAdV DNA,

Providing at least one primer pair that can specifically bind to the DNA of each of at least 25 different HAdV serotypes,

Mixing the at least one primer pair with the sample,

Establishing conditions that allow each one of the primers to specifically bind to one of the DNA strands of every single one of the mentioned 25 HAdV types,

Amplifying the regions—framed by the at least one primer pair—of the DNA of each of the 25 HAdV serotypes actually present in the sample,

Detecting amplified DNA regions.

The primer pair is preferably chosen to specifically bind to the DNA of more than 25 HAdV serotypes, i.e. to the DNA of ≧30, more preferable of ≧40, and even more preferable of all HAdV serotypes.

For this, it is preferable to use as primer nucleic acids according to the invention, in particular those with a sequence of SEQ ID NO. 3 (or complementary thereto) and in particular nucleic acids with (a) the SEQ ID NO. 1 and/or the SEQ ID NO. 2, or (b) a sequence with a homology >78% with respect to SEQ ID NO. 1 or SEQ ID NO 2.

In this, the amplification of the DNA is usually carried out via the PCR process but it is also possible to employ other methods for in vitro amplification of nucleic acid sequences.

The conditions necessary for the primers to specifically bind to the corresponding DNA strands can be established by a person skilled in the art by varying suitable parameters, in particular the temperature.

The detection of the amplified DNA regions may for example be performed by separation on gel with a transilluminator, by concentration determination after re-precipitation (which removes short DNA sections and individual nucleotides from the sample), but also by hybridization with probes, in particular those according to the invention (see above). The above discussion applies accordingly with respect to the more detailed particulars (e.g. order of steps, frequency of individual steps, preparation of samples).

A major advantage of the use of a primer pair—preferably according to the invention—is that it can bind specifically to the DNA of 25, ≧30, ≧40, or even of all HAdV serotypes. Correspondingly, the amplification conditions have to be optimized for only a few primer pairs, ideally only for one primer pair.

The smaller the number of employed primers is, the more precise the control gained over the amplification process, since the respective relevant primer concentration—which affects the melting temperature and other factors—is easier to determine. Moreover, a higher number of primers increases the complexity of the experiment.

More than two primers—forming a first primer pair—are used if the DNA of a second group of HAdV serotypes is to be amplified and/or detected and one or both of the primers of the first primer pair are unable to specifically bind to this DNA. The additional primer or primers has/have to be chosen so that the conditions necessary for it/them to specifically bind to the DNA of the second group of HAdV serotypes also allow the first primer pair to specifically bind to its target DNA.

Additional (appropriately adapted) primers are also used if one requires more stringent conditions, e.g. allowing fewer than four mismatches in the binding of the primer.

Early detection of an infection with (at least) one virus out of a group of HAdV serotypes that is made possible by the invention's method can save a patient's life, in particular for immunodeficient or immunosuppressed patients.

The invention further relates to a method to detect HAdV DNA in a sample, comprising the following steps:

Providing a sample possibly containing HAdV DNA,

Providing at least one primer pair that can specifically bind to each of the DNA of at least 15 different HAdV serotypes,

Providing a probe that can specifically bind—in the regions framed by the at least one primer pair—to the DNA of the same serotype of the at least 15 different HAdV serotypes,

Mixing the at least one primer pair with the sample,

Mixing the probe with the sample,

Establishing conditions that allow each one of the primers to specifically bind to one of the DNA strands of every single one of the mentioned 15 HAdV types.

Amplifying the regions—flanked by the at least one primer pair—of each of the DNA of the 15 HAdV serotypes actually present in the sample.

Establishing conditions that allow the probe to specifically bind to sections of the amplified regions,

Detecting amplified DNA regions to which a probe is bound.

In this it is preferable for the primer pair and the probe to be able to specifically bind not only to the DNA of 15 HAdV serotypes, but to the DNA of ≧20, more preferable of ≧30, even more preferable of ≧40, even more preferable of ≧45, and ideally of all HAdV serotypes. The explanations provided with respect to the above methods are also applicable with respect to the details of the mentioned steps, advantageous primers and probes, and other preferred embodiments of the methods according to the invention.

A particular advantage of simultaneously using a primer pair and a probe, which both can specifically bind to the same DNA of the mentioned number of different HAdV serotypes, is that three specific bindings are necessary and are created for the invention's detection of each HAdV DNA of a multitude of serotypes. This significantly increases the reliability of the detection method compared to methods with only two specific bindings (PCR) or those with only a single specific binding (detection by probe).

Preferred methods according to the invention do not use degenerate primers in the amplification. The exclusive use of non-degenerate primers offers the particular advantage that the reaction behaviour can be calculated more accurately. For example, if the target binding section of the non-degenerate primer is known, it is possible to calculate the annealing behaviour, i.e. the melting temperature, very precisely. For degenerate primers however, if they are produced by the standard oligo synthesis process, one faces the problem that one has no information on the actual ratios of the primer variants that are present. But the concentration of every individual primer in a degenerate primer influences the respective melting temperature, which in turn affects the annealing of an individual primer to the target region of the DNA. In addition, the ratio of the concentrations of the actual primer variants present in a degenerate primer is dependent on the exact manufacturing conditions, which may differ between different suppliers, so that the actual composition of one degenerate primer can differ significantly from that of the “same” degenerate primer supplied by a different manufacturer. Naturally, this has a negative impact on the reproducibility of the detection methods. Moreover, several not precisely defined bases within the degenerate primer give rise to a multitude of possible primer variants, which for the individual primers actually present can mean that they are present in a relatively lower concentration and as a result will underestimate the concentration of the HAdV DNA associated with them or will fail completely in the detection attempt. A further risk related to a large number of primer variants in a degenerate primer is that individual primers could hybridize with each other, which would render them unavailable to the PCR.

Preferred methods according to the invention use fewer than 11, more preferred fewer than 5, and even more preferred fewer than 3 different individual primers in the amplification.

Using a small number of primers improves the computability of the entire annealing and amplification process, in particular if the target sequences (binding regions) on the DNA to be detected are known. Moreover, the above-listed disadvantages of degenerate primers will not apply to primers as per the definition applicable here, not even if several primers are used.

As already mentioned, preferred methods according to the invention employ nucleic acids according to the invention as primers (identical to or derived from SEQ ID NO. 1, SEQ ID NO. 2, or SEQ ID NO. 3, see above) to carry out amplification. These primers to a high degree meet the requirements set for primers in methods according to the invention; in particular they possess the capability of specifically binding to the DNA of a large number of different HAdV sterotypes. In particular, primer pairs for the PCR are selected from deoxynucleic acids according to the invention, whereby a person skilled in the art will have no difficulty to determine—taking into account the synthesis direction of the DNA polymerase—an appropriate sequence for the forward primer and the corresponding one for the reverse primer.

As mentioned, preferred methods according to the invention make use of nucleic acids according to the invention (identical to or derived from SEQ ID NO. 1, SEQ ID NO. 2, or SEQ ID NO. 3, see above) as probes. The person skilled in the art will choose one of SEQ ID NO. 1 to 3 as preferred basis for the probe depending on—among other factors—the DNA region to be amplified.

Particularly preferred in this connection are RNA or DNA probes derived from SEQ ID NO. 3, since for primer pairs derived from SEQ ID NO. 1 and 2, the probe binding section is located in the amplicon. Due to the fact that the two DNA strands that contain the binding region are complementary with respect to each other, it is of secondary importance whether the probe corresponds to one of the above-described sequences or is complementary to it.

The mentioned probes meet the requirements for probes for the invention's methods particularly well; in particular, they are able to specifically bind to the DNA of a large number of HAdV serotypes.

The methods according to the invention are preferably implemented in such a manner so that the amplified region (amplicon) comprises ≦500, preferably ≦300, and more preferably ≦150 base pairs.

The main advantage of an amplicon of small size in comparison to a larger-size amplicon is that the PCR can proceed faster, only a smaller quantity of nucleotides is required, and the accuracy of the DNA amplification is increased.

In particularly preferred embodiments of the methods according to the invention, the detection of the amplified DNA regions is performed under real-time conditions during and/or after one, several, or each amplification step.

“Under real-time conditions” in this context means that the amplification process, which always includes a repeated sequence of several steps (PCR), does not have to be interrupted. In this context, the term “after each amplification step” should not be interpreted as meaning that the detection has to be performed immediately after the completion of the amplification step.

In preferred embodiments of the methods according to the invention, a probe for in-situ detection specifically binds to template DNA and to the DNA of the target HAdV that was amplified in the preceding amplification steps. This preferably takes place during the primer binding step (annealing) during the PCR. The probe must be chosen so that it binds to the target DNA under the same conditions as the primers. Preferred for this is a nucleic acid according to the invention, derived from SEQ ID NO. 3. The binding event can then modify a signal, e.g. intensify or attenuate fluorescence, and can be detected. But the detection also can be carried out on the basis of a reaction that is only facilitated by the binding of the probe to its target section of the DNA, e.g. release of a dye, or by the release of a quencher, e.g. as a result of the nuclease activity of a DNA polymerase.

A method of such design offers the advantage of eliminating the need for labour-intensive and time-consuming steps for detection of the amplicon, such as gel electrophoresis dyed with ethidium bromide with or without restriction enzyme digestion or additional hybridizing procedures.

Furthermore, detection methods that are not performed under real-time conditions, generally require the handling of open PCR products, which presents a high contamination risk: In extreme cases, the carry-over of just a single DNA molecule into subsequent PCR batches can lead to false positive results in the extremely sensitive PCR processes. Such contamination can even occur in an air-borne manner. Accordingly, the more open handling of nucleic acids takes place the more complex will be the safety measures against contamination in labs.

A further advantage of the real-time detection is that in cases where only a qualitative detection of the presence of HAdV DNA is required, the amplification process can be terminated as soon as a signal is present that indicates detection of the corresponding DNA. This can lead to significant time savings.

Moreover, many detection methods employ highly toxic and/or carcinogenic reagents such as ethidium bromide. Thus, detections carried out under real-time conditions also provide improved health protection for the lab personnel. In addition, one saves on costs for special protective equipment, such as protective clothing.

In further preferred embodiments of the above-described methods, the amplified DNA regions are detected quantitatively.

To obtain a relative comparison of the concentrations of detected DNA with respect to each other, a person skilled in the art will use methods such as measuring the UV transmission in a transilluminator after separation in an ethidium bromide dyed gel or the densitometric analysis of images of such gels.

In order to be able to draw conclusions about the absolute DNA concentration in a sample, signals whose strength is directly dependent on the DNA concentration can be compared to standard curves and in this manner be normalized. Particularly suitable for such a process are DNA detection methods that provide signals that can be measured by high-resolution devices. The corresponding signals can for example be provided by probes with radioactive labelling and particularly preferred probes that generate fluorescence or chemiluminescence signals depending on the binding to the DNA, sometimes after additional reaction steps.

Conventional PCRs only provide qualitative results (positive/negative). However, researchers have a need for the quantification of Adenovirus DNA concentrations to obtain insight into the kinetics and pathophysiology of adenovirus infections. A quantitative determination is also necessary in order to obtain fundamental insights into the virus itself, e.g. with respect to virus replication and effect of medication. The methods according to the invention satisfy this need very well, since—different from quantitative methods for HAdV up to now—not only one or a few HAdV serotypes are detected, but rather a larger number (in preferred embodiments of the method even all) of the HAdV serotypes. Thus, methods according to invention designed accordingly are particularly suitable to be used in clinical studies.

Up to now it is impossible to monitor the therapy during the treatment of adenovirus infections (such as for example when applying specific antiviral drugs or the reduction of immunosuppressive therapy). The invention's quantitative detection methods provide a solution to this problem as well, since these methods allow the quantification of the HAdV load of clinical samples—after a suitable preparation thereof—in a highly reproducible manner.

Quantification does not only simplify the monitoring of a therapy's success, but also is instrumental in the decision to commence therapy: In contrast to other methods known to date, the methods according to the invention can not only determine the qualitative fact of a patient's infection with one or several of the large number of HAdV serotypes, but rather can quantify the severity of this viral load. Consequently, in particular for immunosuppressed patients, a physician will only advise a reduction of the suppression if the viral load represents a serious risk for the patient or is expected to do so. And finally, quantification, in particular if used on samples of the same patient taken at different times, facilitates the prediction of the actual outbreak of HAdV-based diseases.

The invention's methods additionally allow the (necessary) quantification of human adenoviruses to aid in the planning and control of gene therapy with adenovirus vectors.

A further, particularly preferred embodiment of the methods according to the invention uses as primers nucleic acids according to the invention, which as described above are homologous with respect to the sequences SEQ ID NO. 1 and/or SEQ ID NO. 2, and/or uses as probe a nucleic acid labeled in accordance with the invention, which as described above is homologous to the sequence SEQ ID NO. 3 or is complementary with respect to such a homologous sequence.

The use of the mentioned primers and/or the mentioned probes provides the advantage that these are primers and probes that are capable of binding to the DNA of a multitude of HAdV serotypes. The other above-mentioned advantages of the invention's nucleic acids will be realized as well when used in a quantification method according to the invention.

For the reasons listed above, a particularly preferred embodiment of the described invention uses as primer (pair) nucleic acids with SEQ ID NO. 1 and SEQ ID NO. 2 and as probe a labeled nucleic acid with SEQ ID NO. 3 or a complementary sequence.

The use of the preferred primers and probes allows the reliable detection of the DNA of all HAdV serotypes in clinical samples, whereby the listed methods according to the invention also allow the quantification—even under realtime conditions—of the HAdV DNA present in the sample. Even though there existed a high demand for such HAdV detection methods, experts in the field were not able to create such a method due to the high sequence diversity of these viruses.

A particularly preferred embodiment of the invention's methods (possibly their preferred implementations) uses a TaqMan PCR process (also known as “exonuclease probe” method) for amplification and detection (see Holland, P. M., Abramson, R. D., Watson, R., and Gelfand, D. H. (1991): Detection of specific polymerase chain reaction product by utilizing the 5′----3′ exonuclease activity of Thermus aquaticus DNA polymerase. Proc Natl Acad Sci USA 88, 7276-80; and Heid, C. A., Stevens, J., Livak, K. J., and Williams, P. M. (1996): Real time quantitative PCR. Genome Res 6, 986-94; sowie Kricka, L. J. (2002): Stains, labels and detection strategies for nucleic acids assays. Ann Clin Biochem 39, 114-29).

Several (partly generic) TaqMan PCR processes for various human pathogenic viruses have been described to date. These methods offer a number of advantages. However, a transfer of this principle to the (simultaneous) detection of a large number of HAdV serotypes has not been achieved to date, on account of the above-mentioned sequence diversity of these viruses, but has now become possible due to the findings of the present invention.

The probe can be labeled in various ways, for example using FAM as fluorescent dye at the 5′ end and TAMRA as fluorescence quencher at the 3′ end. This results in the quenching of the fluorescence of the probe's dye in the unbound state and to the release of fluorescence in the bond state, e.g. by separating the reporter dye and the quencher dye through the 5′-3′ exonuclease activity of the DNA polymerase during the extension step in the PCR.

A particularly preferred embodiment utilizes a probe with SEQ ID NO. 3 and primers with SEQ ID NO. 1 and SEQ ID NO. 2.

A method according to the invention implemented as TaqMan PCR process unites—given a suitable selection of primers—all of the advantages offered by the invention's methods and their preferred implementations:

The amplified DNA is detected under real-time conditions during the PCR by hybridizing the DNA with a probe.

Subsequent detections steps are no longer necessary, which reduces labour and time requirements.

Ease of operation

Contamination of subsequent PCR batches with amplified DNA is almost completely ruled out on account of the TaqMan principle, since no open handling of PCR products is required.

Given an appropriate selection—according to the invention—of primers and probe, the TaqMan PCR method allows the detection of all known HAdV serotypes.

The TaqMan method allows a reliable quantification of the HAdV DNA present in the sample.

Moreover, the TaqMan PCR system is an established method with the necessary components (equipment, chemicals) being commercially available and optimization support easily accessible as well. In particular the quantitative analysis method (Analysis via crossing points, CP) is well established.

An additional advantage of the TaqMan method according to the invention compared to conventional PCR methods is its higher sensitivity. Depending on the implementation of the process, the detection threshold lies at ≦1.5×10⁴ template molecules per batch, preferably at ≦1.5×10³, more preferred at ≦1.5×10², and ultimately preferred at ≦1.5×10¹ template molecules per batch (compare table 1).

In further preferred embodiments of the described methods, the primer annealing takes place at ≧48° C., preferably at ≧50° C., more preferred at ≧53° C., and even more preferred at ≧55° C.

A high (higher) temperature in the annealing phase offers the advantage, of a high (higher) level of certainty that the primer bond is in fact specific.

Part of the invention is a kit that comprises a primer pair and probe, each of which consists of nucleic acids according to the invention.

An essential advantage of such a kit is that the primers and the probe are optimally coordinated and allow implementation of the particularly preferred embodiments of the invention's methods.

The invention also relates to the detection of HAdV DNA using one or several of the invention's nucleic acids or a kit according to the invention. This preferably is done using one of the above-described methods. The results obtained in this manner—or from the prescribed methods—can subsequently form the basis for a physician's diagnosis.

This provides the physician with data that allow him/her to come to well-founded decisions with respect to the necessity and type of therapy for HAdV.

The invention also includes a method to characterize HAdV serotypes, comprising the following steps:

Detection of HAdV DNA in a sample in accordance with one of the above-described methods,

Characterization of HAdV DNA detected in the sample.

A person skilled in the art will know of a number of ways to perform the characterization of detected HAdV DNA. For example, using restriction fragment analysis or particularly via complete or partial sequencing of the DNA in the sample, the DNA can be assigned to one of the six human adenovirus species, or even to a known HAdV serotype.

One possible approach that will be described later is the molecular characterizing of PCR positive samples using Multiplex PCR and sequencing (see example 4).

In the event that DNA of more than one HAdV serotype is present in the investigated sample, it may be necessary to carry out more comprehensive characterizations, in particular to be able to assign the results of the characterization steps to the respective HAdV serotypes. For this, a person skilled in the art can for example employ serotype-specific primers in an additional PCR.

The advantage of the described method is that it not only allows detection of the presence of HAdV but in the subsequent characterization allows the identification of the individual serotypes actually present.

The mentioned method also allows the identification of previously unknown HAdV serotypes, since the methods according to the invention also detect the DNA of unknown HAdV serotypes and consequently can be used in the detection of those serotypes: if the amplified DNA region(s) can not be assigned to any of the already known HAdV serotypes (e.g. in a database comparison after sequencing), the probability is very high that an unknown HAdV serotype is present (unless it is an already known serotype whose base sequence—corresponding to the characterized DNA region—has not yet been entered into the gene database). If necessary, additional characterizations of other DNA regions, preferably of the potential virus itself, can confirm the result that a new HAdV serotype has been discovered.

Another option is to use a method according to the invention to screen the samples for the presence of HAdV DNA and to use conventional techniques to carry out virus isolation and typing for positive samples, e.g. isolation from cell cultures followed in positive cases by typing using neutralization tests, haemagglutination tests, and haemagglutination inhibition tests. New types can not be typed satisfactorily with these conventional techniques and will consequently be revealed as a new non-typeable adenovirus.

Preferred embodiments of the invention will be explained in the following with the help of the figures and the examples.

FIG. 1 represents the multiple alignment of a portion of the hexon gene sequences of various HAdV serotypes, the consensus sequences of the PCR primers (AQ1, SEQ ID NO. 1; AQ2, SEQ ID NO. 2), and of the probe AP (SEQ ID NO. 3). The melting temperatures (Tₘ of the bond of AQ1 and AQ2 as well as AP) to each sequence were determined taking into account the mismatches between the consensus sequence and the respective HAdV sequence. The base numbering is based on the HAdV-2 sequence. The database numbers are: HAdV-2 (#NC 001405), HAdV-3 (#X76549), HAdV-4 (#AF06062), HAdV-5 (#NC 00146), HAdV-12 (AF065065), HAdV-34 (#AB052911), HAdV-40 (#L19443), HAdV-41 (#M21163).

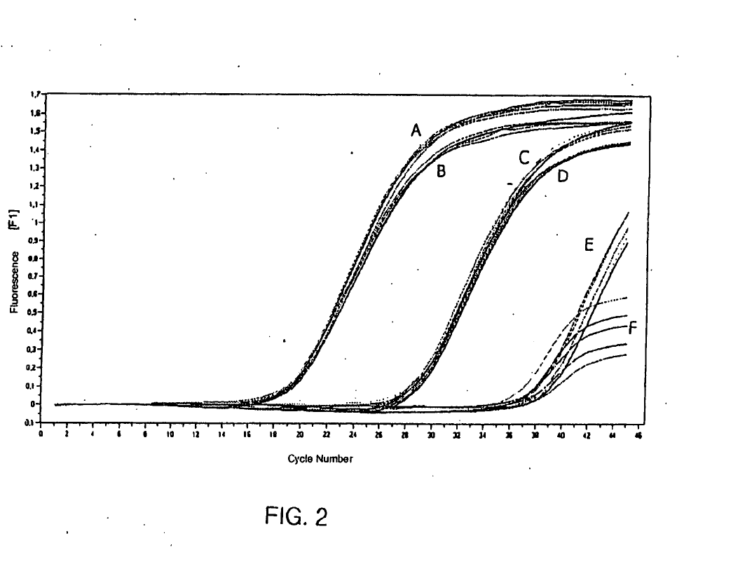

FIG. 2 shows the rise in fluorescence as a function of cycle number. Tested were three different template concentrations (A, B: 1.5×10⁷ copies per run, C, D: 1.5×10⁴ copies per run; E, F: 1.5×10¹ copies per run) with (B, D, F) and without (A, C, E) 500 ng of human DNA. The rise of the fluorescence above the threshold value (Crossing Point, CP) was not affected by the human DNA, whereas the final fluorescence value was.

### EXAMPLE 1

### HAdV Quantification, Standard Plasmid, and Available Viruses

To prepare a standard for positive control, a HAdV-2 PCR amplicon (nt. 18856-19137 of the HAdV-2 sequence) was cloned into a pGEM-T Easy plasmid vector (Promega, Madison, Wis.). The plasmid DNA was purified out of E. coli using the Nukleobond 100 Kit (Macherey and Nagel, Germany) and sequenced to verify that the cloned HAdV-2 sequence was identical to the HAdV-2 prototype Gene bank sequence (#J01917). The plasmid concentration was determined using photometry at 260 nm and converted to genome equivalents (copies per ml), since the molecular weight of the plasmid is known.

For this test series of the HAdV serotypes, A549 cells (Graham 293 cells for HAdV-40 and HAdV-41) were infected with the HAdV prototype strains. For over 50% CPE (cytopathic effect) the cells were freeze-dried and the DNA was extracted from 200 μl of the lysate using the Qiagen Blood Kit (Qiagen, Hilden, Germany). We tested prototype virus strains from the German Nationalreferenz-Laboratorium (Types HAdV-1 to -21, -23, -25, -27, -28, -30 to -41, -43) and from the American Type Culture Collection (Manassas, Va.) (HAdV-3, -5, -7, -12, -18, -22, -24, -26, -29, -30, -35, -36, -42, -44 to -49 and the proposed types -50 und -51).

### EXAMPLE 2

### Design of Primers and Probe

The design process for a primer pair for amplification of the DNA of all 51 serotypes of the genus HAdV was as follows: Five already completely sequenced, type 2 (Species (Genus) human Adenovirus C, Gene bank #J01917), 5 (Species human Adenovirus C, #M73260), 12 (Species human Adenovirus A; #X73487), 17 (Species human Adenovirus D, #AF108105), and 40 (Species human Adenovirus F, Gene bank #L19443) were aligned using the software package clustalX (Version 1.8) (see Thompson, J. D., D. G. Higgins, and T. J. Gibson. 1994. CLUSTAL W: improving the sensitivity of progressive multiple sequence alignment through sequence weighting, position-specific gap penalties and weight matrix choice. Nucleic Acids Res. 22(22):4673-80).

Several highly conserved regions were identified in the hexon gene, whereby these regions not only allow the specific binding of the primers but also that of the labeled probe. Since additional data on the hexon gene sequence of various HAdV types is available, we generated a multiple alignment of the hexon gene with sequences of all six HAdV genera and used this to create the primers (compare FIG. 1). The primers were selected in accordance with the TaqMan PCR guidelines (compare: Sequence detection systems quantitative assay design and optimization; www.appliedbiosystems.com, application note #77101-006) so that the region flanked by the primers contains a third highly conserved region that can serve as binding point for the probe.

In spite of the selection of conserved regions, there exist minor sequence diversities among the binding regions of the primers and the probe. In order to accommodate the maximum number of mismatches, the consensus primer sequences were designed by calculating the melting temperatures of the interaction of the two primers with each HAdV DNA sequence using Metcalc Software (Compare: Schütz, E., and N. von Ahsen. 1999. Spreadsheet software for thermodynamic melting point prediction of oligonucleotide hybridization with and without mismatches. Biotechniques. 27:1218-24.) (see FIG. 1).

An amplification series by a conventional PCR for the DNA of 51 HAdV, which include all serotypes (including the proposed types 50 and 51), has demonstrated that the primer pair (AQ1 and AQ2) is able to amplify DNA regions of all human adenoviruses.

In contrast to a conventional PCR, the real-time detection of the PCR amplicon with a TaqMan probe requires the almost complete hybridization of a double-fluorescence-labeled probe to achieve the degradation of the probe by the nuclease activity of the Taq polymerase. The probe was created in accordance with the guidelines for TaqMan probes (compare: Sequence detection systems quantitative assay design and optimization; www.appliedbiosystems.com, application note #77101-006) and the number of mismatches of the binding to the DNA of each HAdV serotype of the multiple alignment was minimized in a way similar to that used for the primers (compare FIG. 1). The Tₘ of the probe and the primers for hybridization with the corresponding sequence of the DNA of each HAdV serotype was calculated and the reaction conditions of the real-time PCR were set so as to allow the amplification and detection of all (if possible) human pathogenic adenoviruses (compare example 3).

### EXAMPLE 3

### The TaqMan PCR

The TaqMan PCR was carried out in sealed glass capillaries with a total reaction volume of 20 μl using the LightCycler (LC, Roche Diagnostics, Mannheim, Germany). The FastStart Hybridization Kit (Roche) was used to prepare a PCR master mix. As HAdV-specific primers we used the primers Adenoquant 1 (AQ1, SEQ ID NO. 1) and Adenoquant 2 (AQ2, SEQ ID NO. 2). The probe (AP, SEQ ID NO. 3) was labeled with FAM (Carboxyfluorescein) as fluorescent dye at the 5′ end and with TAMRA (Carboxytetramethylrhodamine) as fluorescent quencher at the 3′ end. All oligonucleotides were synthesized, labeled, and purified by Eurogentec (Seraing, Belgium). The probe, the primers, and magnesium chloride were added to the master mix in amounts to achieve these final concentrations: probe 0.4 mM, each primer 0.5 mM, and magnesium chloride 3 mM. Also added to the master mix was thermolabile Uracil DNA Glycosylase (UNG, 1 U/reaction; Roche, Mannheim, Germany). Each capillary was filled with 8 μl of master mix and 12 μl of DNA template solution. The sealed capillaries were centrifuged in a microcentrifuge and placed into the LC.

The reaction conditions were as follows: 5 minutes at 35° C. for the uracil DNA glycosylase incubation, followed by 10 minutes at 95° C. to activate the “hot start” Taq polymerase. 45 cycles were run, each consisting of the denaturation step at 95° C. for 3 seconds, the annealing step at 55° C. for 10 seconds and the extension step at 65° C. for 60 seconds. Between the annealing step and the extension step the temperature was increased at a rate of 0.5° C. per second.

At the end of each extension step, the fluorescence data is recorded in channel F1 (recording mode “single”) of the LC apparatus. The Crossing Point (CP), i.e. the cycle number at which the fluorescence reaches a threshold value, was automatically calculated by the LightCycler Software (Version 3.5c, Settings: proportional base line adjustment, threshold=base line+6 SD of the base line, 2-point set point calculation). After the final cycle, the test tubes were cooled to 30° C. and disposed of without opening the capillaries.

Detection of HAdV Serotypes Using TaqMan PCR:

The real-time PCR (TaqMan PCR) yielded positive test results for all prototype strains of the genus HAdV, including the recently isolated and proposed new types HAdV-50 and HAdV-51. The Crossing Point (CP) values for all prototype strains had low values of <20, which indicates an effective amplification and high sensitivity of the detection. In addition, we used the TaqMan PCR to test twelve clinical isolations of HAdV of different serotypes and all isolations tested positive with low CP values (<20). On the other hand, the real-time PCR with 100 ng of human DNA, which was isolated from cell cultures (MRC5) or from the blood of a healthy adult, always tested negative (n=38).

Sensitivity and Dynamic Range of Quantification:

Plasmid DNA containing a partial sequence of the HAdV-2 hexon gene was serial diluted and used as template for the PCR process (1.5×10⁸ to 1.5×10⁻¹ HAdV-2 genome equivalents (copies) per batch) in ten repeats for each case to test the sensitivity of the TaqMan PCR. While a number as small as 1.5×10¹ copies was reliably detected (n=10), 1.5×10⁰ copies could only be detected occasionally (4 of 10 batches) and even higher dilutions tested negative, just as the negative control batches that contained only distilled water (see table 1).

TABLE 1
Table 1: Test variability of the HAdV TaqMan PCR, determined by
independent batches on different days

| | Proportion | (mean) | | Calculated | |
|---|---|---|---|---|---|
| Genome | of positive | Crossing | | (mean) | |
| equivalent | detections | Point | SD % | concentration | SD % |
| 1.5 × 10⁸ | 10/10 | 15.47 | 4.30 | 1.91E+08 | 15.66 |
| 1.5 × 10⁷ | 10/10 | 19.27 | 4.09 | 1.56E+07 | 13.88 |
| 1.5 × 10⁶ | 10/10 | 22.96 | 3.07 | 1.39E+06 | 15.58 |
| 1.5 × 10⁵ | 10/10 | 26.59 | 2.27 | 1.26E+05 | 11.25 |
| 1.5 × 10⁴ | 10/10 | 29.93 | 2.03 | 1.47E+04 | 32.73 |
| 1.5 × 10³ | 10/10 | 33.37 | 1.77 | 1.49E+03 | 28.13 |
| 1.5 × 10² | 10/10 | 37.23 | 2.03 | 1.22E+02 | 35.13 |
| 1.5 × 10¹ | 10/10 | 39.63 | 2.97 | 2.43E+01 | 49.95 |
| 1.5 × 10⁰ | 4/10 | >41 | nd | nd | nd |
| 1.5 × 10⁻¹ | 0/10 | nd | nd | nd | nd |

nd: not determined

The serial dilution of the plasmid DNA was also used to determine the dynamic range of the quantification. The test variability (standard deviation of CP values) was low, e.g. 2.7% for 1.5×10⁷ copies per batch and und 1.1% for 1.5×10⁴ copies per batch. Table 1 lists the average CP and the standard deviation (SD) of the CPs determined in ten independent batches on different days. A regression analysis of the crossing points against log (HAdV DNA concentration) yielded a very high correlation coefficient (0.99 to 1.0) for a range in concentration between 1.5×10¹ and 1.5×10⁸ copies of HAdV DNA per batch. The resulting regression lines (n=10) have a slope of −3.5 (SD=0.075), which indicates an effective amplification of the adenovirus DNA of the sample (amplification of the DNA by a factor of 1.93 per cycle).

In further experiments, the concentrations of the serial dilution of the plasmid containing HAdV DNA were set as standard and the HAdV DNA concentration of each point was calculated automatically by the LC software (version 3.5c) assuming a semilogarithmic relationship between the crossing points and the HAdV DNA concentration. The calculated HAdV DNA concentrations and the SDs of the calculated concentrations indicate a dynamic range of the HAdV DNA quantification of at least six orders of magnitude (1.5×10⁸ to 1.5×10² copies HAdV DNA); low virus DNA concentrations such as 1.5×10¹ copies can be quantified as well, however the standard deviation is higher (see table 1).

Adding human genomic DNA (500 ng per batch) to the serial dilution of the HAdV template did not affect the sensitivity or quantification of the HAdV DNA detection, as the rise of the fluorescence above the threshold (crossing point value) was unaffected by the human DNA (compare FIG. 2). Unlike the CP values, the endpoint fluorescence data indicate that 500 ng human DNA has a negative effect on the amplification of HAdV DNA (compare FIG. 2).

### EXAMPLE 4

### Characterizing HAdV DNA Present in the Samples Using Molecular Typing

We performed a multiplex PCR, which amplifies the fiber gene region, using positive HAdV DNA samples (after the TaqMan process described in example 3), to allow identification of the respective HAdV serotypes (compare: Xu, W., M. C. McDonough, and D. D. Erdman. 2000. Species-specific identification of human adenoviruses by a multiplex PCR assay. J Clin Microbiol. 38(11):4114-20). We also sequenced amplicons directly using rhodamine-labeled dideoxy nucleotide chain terminators (DNA Sequencing Kit, ABI, Foster City, Calif.) on an ABI-Prism 310 automatic sequencer.

Both the amplification of the hexon gene (compare: Allard, A., B. Albinsson, and G. Wadell. 1992. Detection of adenovirus in stools from healthy persons and patients with diarrhea by two-step polymerase chain reaction. J Med Virol. 37(2):149-57; Xu, W., M. C. McDonough, and D. D. Erdman. 2000. Species-specific identification of human adenoviruses by a multiplex PCR assay. J Clin Microbiol. 38(11):4114-20) and the sequencing allow identification of the HAdV serotype, in some cases with the help of the BLAST and FASTA programs if adequate sequence data is available in gene databases. Since the hexon gene region of many HAdV serotypes has not been sequenced, the sequencing of this region does not allow identification of these HAdV serotypes, but multiple-alignment with database sequences and clustering identification methods allow easy identification at the genus (species) level.

We were able to identify a multitude of HAdV serotypes from clinical samples using the above-mentioned processes (TaqMan PCR, Characterization/Typing) without having to resort to virus isolation. Viruses identified include HAdV of serotypes 1, 3, 4, 5, 7, 37, 40, 41 as well as various viruses of genus HAdV, including one serotype that has not been sequenced to date.

### EXAMPLE 5

### The TaqMan PCR Process in Comparison to Conventional PCR Processes

234 patient samples were assayed using TaqMan (compare example 3) and conventional PCR protocols. After DNA extraction from the samples, the eluate containing the DNA was split for the conventional PCR and the TaqMan PCR. We carried out the conventional adenovirus PCR protocol using the generic primers hex1deg and hex2deg as well as amplification conditions as described in (Allard, A., B. Albinsson, and G. Wadell. 1992. Detection of adenovirus in stools from healthy persons and patients with diarrhoea by two-step polymerase chain reaction. J Med Virol. 37(2):149-57) and (Wadell, G., A. Allard, and H. Hierholzer. 1999. Adenovirus, p. 970-981. In P. R. Murray, E. J. Baron, M. A. Pfaller, C. A. Tenover, and R. A. Yolken (ed.), Manual of Clinical Microbiology. ASM Press, Washington, D.C). The protocol was slightly modified by using a ready-to-use master mix with “hot start” DNA polymerase (Qiagen HotStarTaq Master Mix).

In addition, we examined all samples, for which the results of TaqMan PCR and conventional PCR differed, via a further conventional PCR process using the generic adenovirus primers Ad-1 und Ad-2 as described in (Xu, W., M. C. McDonough, and D. D. Erdman. 2000. Species-specific identification of human adenoviruses by a multiplex PCR assay. J Clin Microbiol. 38(11):4114-20). After amplification, we carried out gel electrophoresis of the PCR products (10 μl) on 2% agarose gels that then were stained with ethidium bromide. Results were visualized by UV illumination.

The 234 clinical samples consisted of EDTA blood (58), serum and plasma (60), throat swabs and/or washes (21), combined nasopharyngeal smears (5), eye smears (17), cerebrospinal fluid (26), stool (22), bronchoalveolar wash and tracheal aspirate (12), as well as 13 other materials, e.g. pericardial, pleural, and peritoneal fluids, urine, and biopsies of lymph nodes and intestine (13).

The results of conventional PCR and TaqMan PCR were identical for 200 samples (38 positive samples and 162 negative samples). For 34 samples, the two test methods yielded different results. In 33 of these samples, the TaqMan PCR yielded positive results with high CP values (CP>37, which means approximately less than 150 copies HAdV DNA per batch), while the conventional PCR yielded negative test results. This shows that the TaqMan PCR process has a higher sensitivity than the described conventional PCR processes.

Only a single sample (EDTA blood) tested positive (narrow bands on the agarose gel) in the conventional PCR process and tested negative in the TaqMan PCR process. This positive result was confirmed using Multiplex PCR and the virus was found to be a virus of the genus HAdV-D. Thus, the described TaqMan PCR process detected the HAdV DNA in 70 of 71 positive samples, while the conventional PCR could only accomplish this in 38 out of 71 positive samples.

The TaqMan process was able to identify individual positive samples from all sample material types, however in the “other materials” group it was only able to make positive identification in samples from lymph node biopsy and from peritoneal fluid (one positive sample each). But the latter does not mean that detection in the other types of samples is fundamentally impossible.

We claim:

1-28. (canceled)

29. A labelled or unlabelled nucleic acid for the specific binding to DNA of human adenoviruses (HAdV DNA), whereby the nucleic acid comprises at least one of the following:
a) the sequence SEQ ID NO. 1 or SEQ ID NO. 3,
b) a sequence with a homology greater than 78% with respect to SEQ ID NO. 1 or SEQ ID NO. 3, or
c) a sequence that is complementary with respect to at least one sequence according to a) or b).

30. A method for the detection of HAdV DNA in a sample, comprising the following steps:
Providing a sample possibly containing HAdV DNA,
Providing a probe that can specifically bind to the DNA of at least 35 different HAdV serotypes,
Mixing the probe with the sample,
Amplifying regions of DNA of each of said at least 35 HAdV serotypes actually present in the sample, so that the section to which said probe can specifically bind is amplified as well,
Establishing conditions that allow the probe to specifically bind to sections of the amplified regions,
Detecting the amplified DNA regions to which a probe has bound, quantitatively and/or under real-time conditions.

31. A method for the detection of HAdV DNA in a sample, comprising the following steps:
Providing a sample possibly containing HAdV DNA,
Providing at least one primer pair that can specifically bind to the DNA of at least 25 different HAdV serotypes,
Mixing the at least one primer pair with the sample,
Establishing conditions that allow one of the primers to specifically bind to one of the DNA strands of every single one of said at least 25 HAdV serotypes,
Amplifying regions of the DNA of each of said at least 25 HAdV serotypes actually present in the sample, wherein said regions of the DNA are flanked by the at least one primer pair,
Detecting amplified DNA regions, quantitatively and/or under real-time conditions.

32. A method for the detection of HAdV DNA in a sample, comprising the following steps:
Providing a sample possibly containing HAdV DNA,
Providing at least one primer pair that can specifically bind to the DNA of at least 15 different HAdV serotypes,
Providing a probe that can specifically bind—in the region flanked by at least one said primer pair—to the DNA of said at least 15 different HAdV serotypes,
Mixing the at least one primer pair with the sample,
Mixing the probe with the sample,
Establishing conditions that allow one of the primers to anneal to one of the DNA strands of every single one of said at least 15 HAdV types,
Amplifying the regions of the DNA of all of said at least 15 HAdV serotypes actually present in the sample, wherein said regions of the DNA are flanked by the at least one primer pair,
Establishing conditions that allow the probe to specifically bind to sections of the amplified regions,
Detecting amplified DNA regions to which a probe has bound, quantitatively and/or under real-time conditions.

33. The method according to one of claims 30, 31 or 32, wherein the amplification uses a primer that comprises at least one of the following:
a) the sequence SEQ ID NO. 1,
b) a sequence with a homology greater than 78% with respect to SEQ ID NO. 1, or
c) a sequence that is complementary to at least one of the nucleic acids according to a) or b).

34. The method according to claim 33, wherein the amplification further uses a primer that comprises at least one of the following:
a) the sequence SEQ ID NO. 2,
b) a sequence with a homology greater than 78% with respect to SEQ ID NO. 2, or
c) a sequence that is complementary to at least one of the nucleic acids according to a) or b).

35. The method according to one of claims 30, 32, 33 or 34, wherein said probe comprises a nucleic acid comprising at least one of the following:
a) the sequence SEQ ID NO. 3,
b) a sequence with homology greater than 78% with respect to SEQ ID NO. 3, or
c) a sequence that is complementary to at least one of the nucleic acids according to a) or b).

36. The method according to one of claims 30, 31, 32, 33, 34 or 35, wherein a TaqMan PCR process is used for amplification and detection.

37. A kit, comprising one primer pair, said primer pair consisting of two primers, wherein said two primers comprise at least one of the following:
a) the sequence SEQ ID NO. 1 and SEQ ID NO. 2, respectively
b) a sequence with homology of greater than 78% with respect to SEQ ID NO. 1 and SEQ ID NO. 2, respectively, or
c) a sequence that is complementary to at least one of the nucleic acids according to a) or b), and further comprising one probe, said probe comprising at least one of the following:
d) the sequence SEQ ID NO. 3,
e) a sequence with homology greater than 78% with respect to SEQ ID NO. 3, or
f) a sequence that is complementary to at least one nucleic acid according to d) or e).

38. A method to characterize HAdV serotypes, comprising the following steps:
Detecting HAdV DNA in a sample in accordance with one of claims 30, 31, 32, 33, 34, 35, or 36, and
Characterizing detected HAdV DNA that is present in the sample.


SEQUENCE LISTING (3 sequences)

SEQ ID NO: 1 - DNA, 23 bp, Artificial
  Consensus sequence for HAdv
gccacggtggggtttctaaactt

SEQ ID NO: 2 - DNA, 25 bp, Artificial
  Consensus sequence for HAdv.
gccccagtggtcttacatgcacatc

SEQ ID NO: 3 - DNA, 29 bp, Artificial
  Consensus sequence for HAdv.
tgcaccagacccgggctcaggtactccga